(19) United States
(12) Patent Application Publication
(10) Pub. No.: US 2015/0297369 A1
(43) Pub. Date: Oct. 22, 2015

(54) PROSTHETIC LINER

(71) Applicant: OTTO BOCK HEALTHCARE GMBH, Duderstadt (DE)

(72) Inventors: Luder Mosler, Duderstadt (DE); Kai Bussiek, Gottingen (DE)

(73) Assignee: OTTO BOCK HEALTHCARE GMBH, Duderstadt (DE)

(21) Appl. No.: 14/443,887

(22) PCT Filed: Nov. 16, 2013
(86) PCT No.: PCT/EP2013/003459

§ 371 Date: May 19, 2015

(30) Foreign Application Priority Data
Nov. 19, 2012 (DE) 10 2012 022 484.2

(51) Int. Cl. A61F 2/78

(52) U.S. Cl. CPC A61F 2/7812; A61F 2002/7818; A61F 2230/0067

(57) ABSTRACT

A prosthetic liner for forming an interface between a prosthesis socket and a residuum to be accommodated in the prosthesis socket. The prosthetic liner including a proximal region with a proximal end and a distal region with a distal end and being provided with a closed-cross section with a proximal opening through which the residuum in inserted into the prosthetic liner. The internal contour has at least on one lateral side of the prosthetic liner a greater pitch from distal to proximal in the proximal region than in the distal region.

The invention relates to a prosthetic liner for forming an interface between a prosthesis socket and a stump to be accommodated in the prosthesis socket, the prosthetic liner having a proximal region with a proximal end and a distal region with a distal end and being provided with a closed cross section with a proximal opening through which the stump is inserted into the prosthetic liner.

To fit patients with a prosthesis, it is necessary to provide a safe and load-bearing arrangement and binding of the prosthetic device on the patients. For this purpose, prosthetic devices have a stable prosthesis socket which more or less exactly simulates the contour of the stump. For this purpose, it is necessary to use a model of the stump, or to use computer data, in order to provide a socket that fits as exactly as possible.

Instead of binding the prosthesis socket directly to the stump, an alternative technique is one in which a liner made of an elastic material, for example silicone, is applied to the stump. The liner is rolled onto the stump and serves to compress and protect the soft-tissue part of the stump. The inside of the liner, i.e. the inner surface of the liner facing the stump, can be made adhesive. A communicating holding system holds the stump/liner combination in the prosthesis socket, for example by means of a vacuum. It is likewise possible, and provision is made for this, that locking pins are provided at the distal end of the liner, by means of which locking pins the liner can be locked mechanically to the prosthesis socket.

A multi-layer prosthetic liner is known from US 2009/0240344 A1. The liner has a round cross section perpendicular to the longitudinal extent and is closed and cap-shaped at the distal end, wherein the liner wall extends in a straight line from the distal end and widens conically. Such a liner is relatively easy to produce in large batch numbers.

When using such a liner, the problem that arises is that there is often inadequate adherence of the liner to the stump on the lateral side.

The object of the present invention is therefore to make available a prosthetic liner which, while providing good adherence to patients, at the same time has sufficient durability and in addition does not require individual preparation.

According to the invention, this object is achieved by a prosthetic liner having the features of the main claim. Advantageous embodiments and developments of the invention are disclosed in the dependent claims, the description and the figures.

The prosthetic liner according to the invention for forming an interface between a prosthesis socket and a stump to be accommodated in the prosthesis socket, the prosthetic liner having a proximal region with a proximal end and a distal region with a distal end and being provided with a closed cross section with a proximal opening through which the stump is inserted into the prosthetic liner, is characterized in that the internal contour, at least on a lateral side of the prosthetic liner, has a greater pitch from distal to proximal in the proximal region than in the distal region. Whereas, in the case of a cylindrical configuration of a prosthetic liner, at least in the proximal region, there is a danger of tears occurring on the medial side, and, in the case of a conical configuration of a liner, a danger of a poor fit on the lateral side, the design of the prosthetic liner according to the invention provides good adherence both on the medial side and also on the lateral side, with at the same time good durability of the liner in the proximal region. The increasing pitch or curvature in the direction of the stump from the distal end toward the proximal end has the advantage that an internal contour and thus also an external contour that follows the contour of the body is obtained on the lateral side, in particular in the area of the hip joint in prosthetic liners for thigh stumps. The different pitches in the distal region and proximal region are in particular also provided in the state when not fitted. It is enough if, in the proximal region, only one area has a greater pitch than the distal region, in order thereby to effect a curvature and orientation in the direction of the stump. The greater pitch or curvature in the direction of the stump does not have to be present over the entire proximal region; in particular the pitch or curvature does not have to increase in the proximal region, and it is instead enough if the pitch or curvature or orientation in the direction of the stump is greater than in the distal region in the unfitted state. Pitch is understood here as the ratio of the difference of the Y values to the associated difference of the X values in a profile of the contour in a Cartesian system of coordinates. The pitch of the contour is then zero if the tangent to the contour is horizontal, wherein the Y axis corresponds substantially to the longitudinal extent of the liner and is oriented from proximal to distal. The greater the pitch, the more the profile of the contour approximates to the perpendicular. A curvature of the contour beyond the perpendicular in the direction of the stump, i.e. into the receiving space for the stump formed by the liner, is regarded as an increasing curvature when the curvature is viewed from distal to proximal. The greater pitch signifies at the same time a greater curvature in the direction of the stump, starting from the distal end of the liner and extending in the direction of the proximal end.

In the distal region, the prosthetic liner can have a conical or parabolic internal and/or external contour, i.e. after the cap-like end it can have a substantially uniform pitch in the case of a conical contour, which then merges in the proximal region into an increasing pitch of curved or cylindrical internal contour and thus generally also external contour. It is likewise possible that a parabolic contour is already present in the distal region, resulting in a change of pitch extending over the entire distal region.

In the proximal region, a cylindrical, i.e. substantially rectilinear, or parabolically curved internal contour can be present, such that the increase in pitch continues from the distal region also into the proximal region and can also have an inward curvature in the direction of the stump beyond the perpendicular.

The distal region, seen from the distal end, can make up over 30% to 75% of the total length of the prosthetic liner. The total length is to be understood as the maximum length of the prosthetic liner from the distal end to the proximal end. There is the possibility that the prosthetic liner, at its proximal end, extends obliquely with respect to the longitudinal extent, which is to be expected especially in liners for thigh stumps, since the medial side should extend only as far as the perineum, while the lateral side should extend as high as possible to the hip in order to permit a sufficient support for the prosthesis socket.

The prosthetic liner can have a multi-layer configuration at least in the distal region and be made of different materials. Preferably, an inner layer of silicone and an outer layer of polyurethane are provided, since silicone has good skin compatibility, and since polyurethane as the outer layer can provide a good distribution of pressure and resistance to wear. Silicone permits very good adherence of the prosthetic liner to the stump, while at the same time having good skin compatibility, and the polyurethane layer on the outside of the prosthetic liner permits adaptation to the prosthesis socket. In principle, it is also possible to use another material pairing, for example with silicone on the outside and a copolymer on the inside, or vice versa.

A textile reinforcement can be provided on or in the prosthetic liner, so as to be able to provide increased dimensional stability and volume stability. The textile reinforcement can extend over the entire liner; provision is likewise made that the textile reinforcement is arranged only in some areas in the prosthetic liner, for example only in the distal region or in the proximal region. The textile reinforcement can be embedded in a layer of the prosthetic liner, incorporated between two layers, for example an inner layer and an outer layer of different materials, and can be completely covered by these layers or can be arranged on a surface of the prosthetic liner. For example, the arrangement on the outside of the prosthetic liner can be provided in order to achieve an optimized material pairing with a corresponding coating on the inside of the prosthesis socket.

A discontinuous change of pitch from the distal end to the proximal end of the prosthetic liner may be present in order to be able to adapt the contour of the liner as correctly as possible to the respective stump. The discontinuous pitch means that the increase of the pitch from the distal region to the proximal region takes place in steps, wherein the pitch increases in each case from distal to proximal and merges into an internal curvature. There is likewise the possibility that the pitch increases from distal to proximal between the discontinuous regions.

The liner is preferably designed closed at the distal end, such that the distal end of the stump is covered by the liner material.

To increase the wearing comfort of the liner, an outward bulge can be provided at the proximal end on the medial side, such that the upper edge, i.e. the proximal edge, nestles against the perineum in the medial region on account of the outward bulge. The bulge at the proximal edge can also be formed circumferentially.

The prosthetic liner can be shorter on the medial side than on the lateral side, in order to achieve higher coverage in the proximal direction on the lateral side, such that, in combination with the curvature in the direction of the body or increased curvature in the direction of the body or stump, good contact with the stump can be achieved on all sides along the entire length of the prosthetic liner.

The prosthetic liner is preferably rotationally symmetrical with respect to its longitudinal axis, at least on its outside, and is made of an elastic material so as to permit easy adaptation of the prosthetic liner to the approximately rotationally symmetrical stump. The rotationally symmetrical design of the prosthetic liner permits simple, cost-effective and rapid production and simple adaptation to prefabricated prosthesis sockets. There is the possibility that the internal contour of the prosthetic liner has, at least on the lateral side, a profile deviating from the contour of the medial side, for example in order to permit an improved contact with the stump. The prosthetic liners are preferably produced in standard sizes, their length being sufficient for all conceivable stumps. The variation in size relates mainly to the diameter or circumference of the prosthetic liner and, if appropriate, to the curvature or pitch of the contour. To adapt the prefabricated prosthetic liners to the respective patients, provision is made to shorten their length and cut the proximal edge to size such that it meets the needs of the patient, i.e. is shortened in particular in the medial region and left as long as possible in the lateral region. The prosthetic liner according to the invention is thus designed as a prefabricated standard liner which does not have to be adapted individually to the respective external contour of a stump, and instead a liner can be chosen from a limited number of models and sizes and can be adapted to the patient by simple modifications.

In one variant of the invention, a padding or thickening is present at the distal region of the prosthetic liner, in order to achieve improved protection of the stump against pressure loads. The padding or thickening can be formed on the outside of the prosthetic liner by applying a material to an outer layer of a prefabricated standard liner, for example by gluing or integral bonding by crosslinking of uncrosslinked polymers. It is also possible for the material thickening to be formed in one piece with the prosthetic liner, i.e. formed directly thereon, for example by shaping during casting or dipping of the liner. The padding or material thickening is arranged in particular on the lateral side of the prosthetic liner, wherein this orientation is present in the fitted state of the liner.

Illustrative embodiments of the invention are explained in more detail below with reference to the attached figures. Identical reference signs indicate identical structural elements. In the drawing:

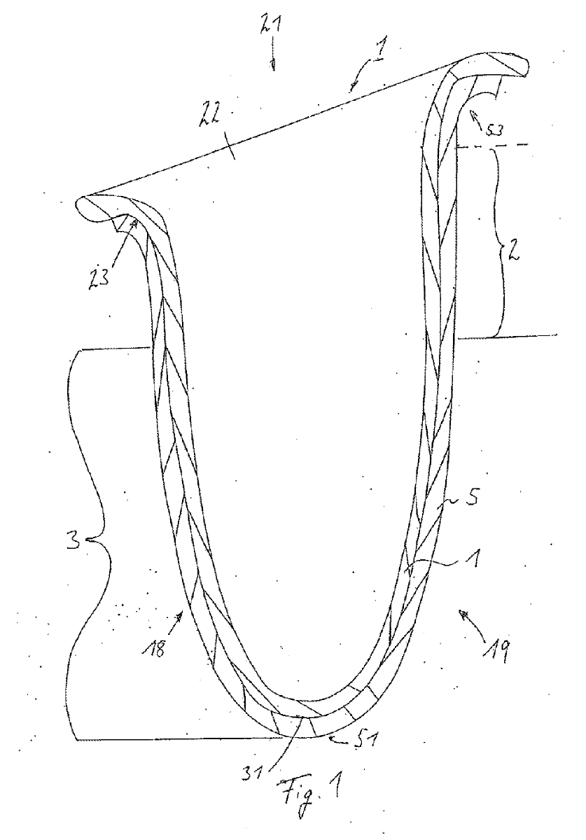

FIG. 1 shows a schematic sectional view of a prosthesis socket with a liner;

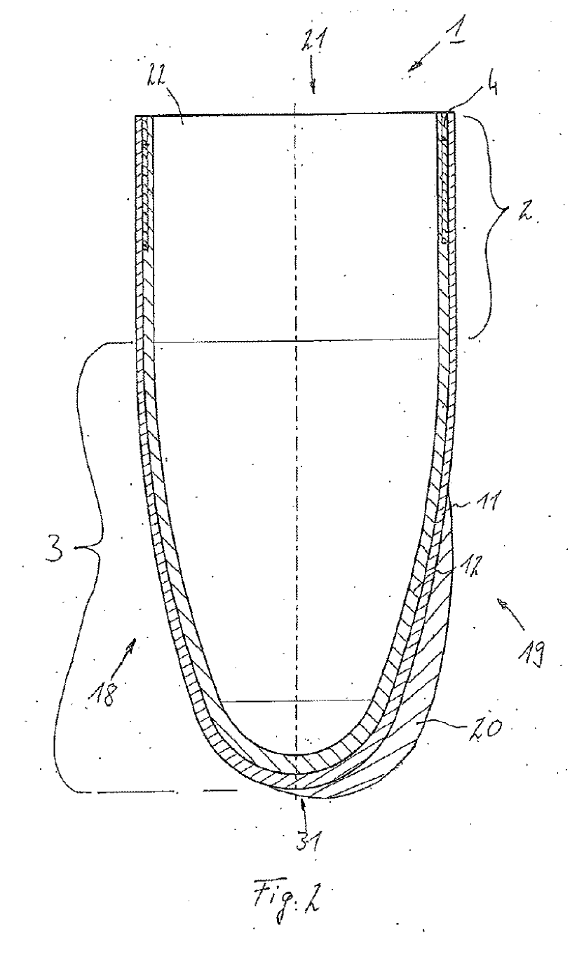

FIG. 2 shows a liner in a sectional view; and

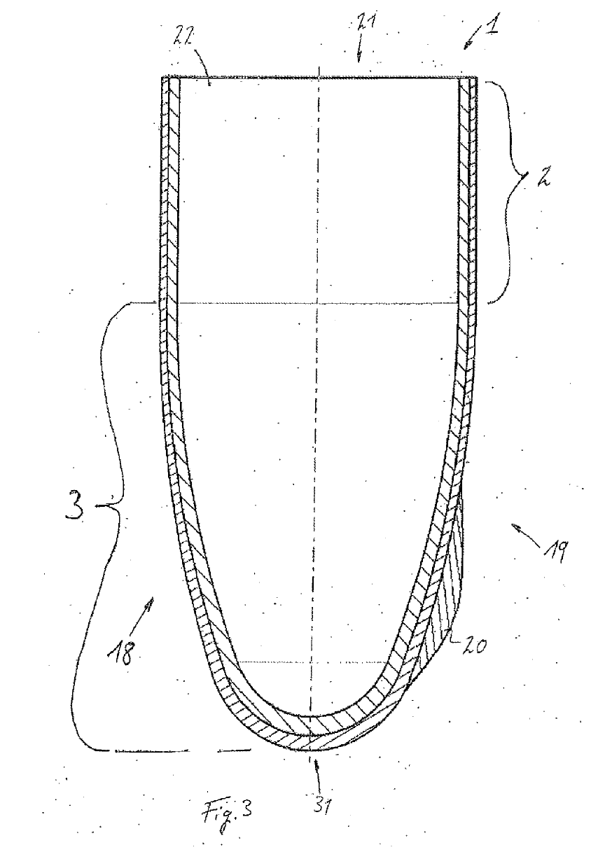

FIG. 3 shows a variant of the prosthetic liner.

In FIG. 1, a prosthesis socket 5 made of a dimensionally stable material is shown schematically in a sectional view. The prosthesis socket 5 is a substantially tubular construction and has an internal contour approximated to the external contour of a stump (not shown), for example a thigh stump, that is to be accommodated therein. In order to achieve sufficient volume stability, the prosthesis socket 5 is designed closed in the circumferential direction and, at its distal end 51, can have a through-hole through which, for example, a locking pin can be guided. Further prosthesis components can be provided at the distal end 51, for example an adapter for receiving joint devices or, in the case of prosthetic legs, a below-knee tube and a prosthetic foot. At the proximal end of the prosthesis socket 5 lying opposite the distal end 51, an insert opening is provided through which the stump (not shown) can be guided.

In the illustrative embodiment shown, a prosthetic liner 1 is arranged inside the prosthesis socket 5. No free space is present between the external contour of the prosthetic liner 1 and the internal contour of the prosthesis socket 5. When the prosthetic liner 1 and the prosthesis socket 5 are both fitted in place, the external contour of the prosthetic liner 1 bears on the internal contour of the prosthesis socket 5 as far as possible across the whole surface. When fitted, the prosthetic liner 1 forms the interface between the stump and the prosthesis socket 5.

In the illustrative embodiment shown, the prosthetic liner 1 arranged inside the prosthesis socket 5 is constructed as a one-layer prosthetic liner 1, which can be made of a silicone, a copolymer or polyurethane. It is likewise possible to provide a multi-layer prosthetic liner 1, which has at least an inner layer and an outer layer of different materials such as silicone and polyurethane or a copolymer, if appropriate with an intermediate layer and/or an insert made of another plastic or a textile or fiber material. The prosthetic liner 1 has a proximal end 21 and a distal end 31. In the illustrative embodiment, the distal end 31 is designed closed, resulting in a cap-like structure on the distal endpiece of the prosthetic liner 1. At the proximal end 21 of the prosthetic liner 1, an opening 22 is formed for inserting a stump.

In the variant of the invention according to FIG. 1, the prosthesis socket 5 and the prosthetic liner 1 have a shorter length on the medial side 18 than on the lateral side 19. In the illustrative embodiment shown, the section contour along the longitudinal extent provides two different regions, namely a proximal region 2 and a distal region 3. The proximal region 2, which extends from the upper, proximal edge 21 of the prosthetic liner 1 in the direction of the distal end 31 thereof, is of substantially cylindrical construction. The pitch of the contour in the proximal region 2 is thus substantially constant, whereas the internal contour of the prosthetic liner 1 curves in the distal region. Where seen from the distal end 31 of the prosthetic liner 1, the internal contour initially has no pitch, since the tangent to the distal end 31 extends horizontally. In the further course from distal to proximal, the pitch within the distal region 3 increases until, in the transition to the proximal region 2, an at least cylindrical internal contour is present. Provision is also made that the internal contour of the prosthetic liner 1 curves further in the direction of the stump in order to permit an improved adherence of the liner, in particular on the lateral side 19. The proximal end of the proximal region 2 can be adjoined by an end region with an outward bulge. To increase the wearing comfort, the proximal region 2 is larger on the lateral side 19 than on the medial side 18. Moreover, an outward bulge 23, 53 is arranged on the medial side 18 and on the lateral side 19 at the upper edge of the prosthetic liner 1 and of the prosthesis socket 5, such that, in the case of a thigh liner, this upper edge on the medial side 18 can bear on the perineum, as a result of which the wearing comfort is likewise increased.

The prosthetic liner 1 arranged inside the prosthesis socket 5 has a rotationally symmetrical construction along the longitudinal extent, at least as far as the proximal end of the medial proximal region 2.

FIG. 2 shows a view of the prosthetic liner 1 on its own. The prosthetic liner 1 has an outer layer 11, and an inner layer 12 secured on the outer layer 11. Both layers 11, 12 are permanently connected to each other. The inner layer 12 is preferably made of silicone or a silicone material, while the outer layer 11 is made of a copolymer, if appropriate of polyurethane. A textile reinforcement 4 is arranged between the outer layer 11 and the inner layer 12 and is embedded completely in the prosthetic liner 1, i.e. covered completely on the outer side and inner side, and only possibly reaches the surface at the proximal end. The textile reinforcement 4 is of substantially cylindrical construction and, in the illustrative embodiment shown, is arranged in a proximal portion of the prosthetic liner 1. In principle, it is also possible that the textile reinforcement 4 is arranged along the entire length of the prosthetic liner 1, and several mutually independent textile reinforcements 4 can also be arranged and embedded inside the prosthetic liner 1. The textile reinforcements 4 can have different degrees of elasticity, such that different degrees of stretching can be set along the length of the prosthetic liner 1 while the load remains the same. It is possible in principle, and provision is made for this, that textile reinforcements 4 can also be arranged in one-layer prosthetic liners 1.

In principle, the prosthetic liner can also have a matrix that prevents or minimizes stretching in the longitudinal extent and permits stretching in the circumferential direction. It is thereby possible to obtain anisotropic stretching for the prosthetic liner. The matrix can be arranged as a fabric or other insert in or on the prosthetic liner, preferably in the distal region of the prosthetic liner. The matrix, insert or reinforcement prevents or minimizes the so-called milking effect which, if the prosthetic liner is locked distally to the prosthesis socket, can occur as a result of alternating stretching in the direction of the longitudinal extent of the prosthetic liner.

To fit the prosthetic liner 1 in place, the prosthetic liner is first of all wound up from the direction of the proximal end, the distal end 31 is placed with the inner layer 12 onto the stump, and the prosthetic liner 1 is then rolled over the stump such that the inner layer 12 lies on the stump and covers the latter as far as possible over the whole surface. The outer layer 11 serves to provide a large area of advantageously adhesive contact with the inner surface of the prosthesis socket 5, which is not shown in FIG. 2.

The prosthetic liner 1 is of rotationally symmetrical construction along its longitudinal extent, i.e. from the distal end 31 to the proximal end 21, in other words it basically has a circular cross section perpendicular to the longitudinal extent at least on the outside.

In the direction toward the distal end 13, the proximal region 2 is adjoined by a distal region 3, which extends as far as the distal end 31 of the prosthetic liner 1. Starting from the distal tip, at which the pitch of the contour is zero, the pitch of the contour of the outer side and inner side of the prosthetic liner 2 increases continuously as far as the proximal region 2, such that the distal region 3 has a substantially parabolic internal contour. The increase of the pitch is identical on the medial side 18 and on the lateral side 19, resulting in a symmetrical configuration of the prosthetic liner 1. For adapting the prosthetic liner 1 to the patient, it is thus possible to fit the prosthetic liner 1 without paying heed to a particular orientation relative to the stump.

By configuring the prosthetic liner 1 with a contour which at least on the lateral side has a pitch that is greater in the proximal region 2 than in the distal region 3, it is ensured that particularly good adherence on the stump is obtained at the upper edge of the prosthetic liner 1. The inward prestressing obtained by the inward bulge of the liner 1 ensures that there is no free space between the stump and the liner. A negative pitch can also be set in the proximal region 2. In particular in the beveled variant according to FIG. 1, it is ensured that excessive stress, which leads to tears in the liner material, is not built up on the medial side 18. After the prosthetic liner 1 has been adjusted to the necessary length, the desired inward curvature is present at least on the lateral side 19. A limited number of lengths and diameters is generally sufficient to be able to fit the stump shapes that usually arise.

On the lateral outer face of the prosthetic liner 1, a padding 20 is provided in the distal region 3 and extends from the distal end 3 of the prosthetic liner 1 to beyond the center of the distal region 3 in the direction of the proximal region 2. In the illustrative embodiment shown, the padding 20 is arranged only on the lateral side 19, such that the rotational symmetry is no longer present. The initially rotationally symmetrical prosthetic liner 1 with the inner layer 12 and the outer layer 11 was provided subsequently with the padding 20, which was secured on the outer face of the outer layer 11, for example adhesively bonded or permanently secured by crosslinking with the material of the outer layer 11. The material of the padding 20 can differ from the material of the outer layer 11. As an alternative to the illustrative embodiment shown, it is also possible that the padding 20 is arranged rotationally symmetrically about the entire outer surface at least of the distal region 3. It is likewise possible to make the padding 20 from the material of the outer layer 11 and to form the outer layer 11 in one piece, such that the padding 20 is already generated in the production of the outer layer 11. Particularly in the case of mature atrophied stumps with low soft-tissue coverage, the padding 20 in the lateral femoral region permits artificial soft-tissue padding. Moreover, with the femur in an abducted position, the padding permits an additional positioning of the femur, with the latter being moved inward, i.e. in the medial direction. The positioning of the femur in the prosthesis socket is thus supported. When the prosthetic liner 1 is designed as a thigh liner, the padding 20 thus effects a soft-tissue padding in the region of the femur and a positioning of the femur and reduces the loads on the thigh bone.

The arrangement of the padding 20 at the distal end 31 of the prosthetic liner 1 provides a material thickening such that, in the event of an axial load, i.e. a load along the longitudinal extent of the prosthetic liner 1, an improved cushioning effect is obtained.

FIG. 3 shows a variant of the invention in which the padding 20 is arranged exclusively on the lateral side of the prosthetic liner. Thus, only a relatively small area of the distal region 3 located on the lateral side 19 is provided with the padding 20, so as to provide a material thickening specifically at those places that have to be specially protected. When fitting the liner in place, it is simply a matter of precisely positioning the padding 20 at the desired position, preferably on the lateral side of the stump.

The prosthetic liner 1 according to the embodiment in FIG. 3 has no textile insert 4 or any reinforcement in the intermediate area between the inner layer 12 and the outer layer 11. In the two embodiments of the liner 1 according to FIGS. 2 and 3, the proximal region 2 extends as far as the proximal end 21, wherein the internal contour has, on account of the cylindrical configuration, a greater pitch than the contour of the distal region 3. In principle, it is also possible that a further curvature can be provided in the direction of the stump (not shown) or the center axis of the prosthetic liner 1, at least on the lateral side 19, in order to effect an optimized adherence and contact of the prosthetic liner 1 on the external contour of the stump.

In the embodiments in the figures, the distal region 3 is conical or parabolic and extends over more than half of the total length of the liner. The proximal region 2 merges into a cylindrical or approximately cylindrical shape and forms the remaining length for the prosthetic liner 1. As a result of the contour of the prosthetic liner 1 widening in the proximal direction on the medial side 18, the occurrence of tears in the medial edge area is avoided. In the embodiments shown, the prosthetic liner 1 is designed closed at the distal end and has a closed circumference, such that it can also be used as a liner in suction sockets. Moreover, the liner can be used in prosthesis sockets that are used as a pure volume socket without osseous tuber support, as a result of which the distal portion of the stump has to obtain an enhanced wearing function. It is therefore advantageous if a soft, adhesive inner layer 12 of silicone and a stable and adhesive outer layer 11 of polyurethane or of another copolymer are present. The distal region 3 can have a greater wall thickness than the proximal region 2, and in particular the distal end 31 of the prosthetic liner 1 can have an increased material thickness, in particular of the inner layer 12.

The cross section of the prosthetic liner is designed closed in a direction perpendicular to its longitudinal extent, thus resulting in a substantially tubular shape. The distal end of the liner can also be designed open, such that it is not a closed receptacle with a proximal opening that is obtained, but instead a tube open at both ends and with a corresponding internal contour.

1. A prosthetic liner configured to form an interface between a prosthesis socket and a stump to be accommodated in the prosthesis socket, the prosthetic liner comprising:
a proximal region with a proximal end;
a distal region with a distal end;
a closed cross section with a proximal opening through which the stump is inserted into the prosthetic liner;
wherein an internal contour, at least on a lateral side of the prosthetic liner, has a greater pitch from distal to proximal in the proximal region than in the distal region.

2. The prosthetic liner as claimed in claim 1, wherein the distal region has a conical or parabolic contour along at least one of an internal or external surface thereof.

3. The prosthetic liner as claimed in claim 1, wherein the proximal region has a cylindrical or parabolically curved internal contour.

4. The prosthetic liner as claimed in claim 1, wherein the distal region, seen from the distal end, extends over 30% to 75% of a total length of the prosthetic liner.

5. The prosthetic liner as claimed in claim 1, wherein the prosthetic liner has a multi-layer configuration at least in the distal region and has an inner layer made of a material different than a material of an outer layer.

6. The prosthetic liner as claimed in claim 1, wherein a textile reinforcement is provided in the prosthetic liner.

7. The prosthetic liner as claimed in claim 1, wherein there is a discontinuous change of pitch from the distal end to the proximal end.

8. The prosthetic liner as claimed in claim 1, wherein the prosthetic liner is designed closed at the distal end.

9. The prosthetic liner as claimed in claim 1, wherein the prosthetic liner is shorter on the a medial side than on the lateral side.

10. The prosthetic liner as claimed in claim 1, wherein the prosthetic liner is designed rotationally symmetrically in a longitudinal extent.

11. The prosthetic liner as claimed in claim 1, wherein a padding is present in the distal region, on at least the lateral side.

12. The prosthetic liner of claim 5, wherein the inner layer comprises silicone.

13. A prosthetic liner, comprising:
a proximal region having a proximal end and a proximal opening through which a stump is inserted into the prosthetic liner;
a distal region having a closed distal end;
an inner surface having an internal contour with a greater pitch in the proximal region than in the distal region along at least on a lateral side of the prosthetic liner, the pitch being measured in a direction toward the proximal end.

14. The prosthetic liner as claimed in claim 13, wherein the distal region has a conical or parabolic contour along at least one of the internal surface or an external surface thereof.

15. The prosthetic liner as claimed in claim 13, wherein the proximal region has a cylindrical or parabolically curved internal contour.

16. The prosthetic liner as claimed in claim 13, wherein the distal region, viewed from the distal end, extends over 30% to 75% of a total length of the prosthetic liner.

17. The prosthetic liner as claimed in claim 13, wherein the prosthetic liner has a multi-layer configuration at least in the distal region, the multi-layer configuration including an inner layer made of a material different than a material of an outer layer.

18. The prosthetic liner as claimed in claim 13, wherein a textile reinforcement is provided in the prosthetic liner.

19. The prosthetic liner as claimed in claim 13, wherein there is a discontinuous change of pitch from the distal end to the proximal end.

20. The prosthetic liner as claimed in claim 13, wherein the prosthetic liner is shorter on a medial side than on the lateral side.
